(19) United States
(12) Patent Application Publication
(10) Pub. No.: US 2010/0190270 A1
(43) Pub. Date: Jul. 29, 2010

(54) SYSTEM AND METHODS FOR DETECTING A GASEOUS ANALYTE IN A GAS

(75) Inventors: Gianluca Piazza, Philadelphia, PA (US); Alan T. Johnson, Philadelphia, PA (US); Matteo Rinaldi, Philadelphia, PA (US); Chiara Zuniga, Philadelphia, PA (US)

Correspondence Address: RATNERPRESTIA, P.O. BOX 980, VALLEY FORGE, PA, 19482, US

(73) Assignee: The Trustees of the University of Pennsylvania, Philadelphia, PA (US)

(21) Appl. No.: 12/693,046

(22) Filed: Jan. 25, 2010

Related U.S. Application Data
(60) Provisional application No. 61/146,866, filed on Jan. 23, 2009.

(51) Int. Cl. G01N 33/551; G01N 29/02; B05D 5/12; H01L 41/22

(52) U.S. Cl. 436/524; 73/24.06; 427/100; 216/13; 977/742; 977/843

(57) ABSTRACT

Systems and methods for detecting a gaseous analyte utilize a micromechanical piezoelectric resonator having a functionalization layer configured to bind with the gaseous analyte. The functionalization layer may include a layer of carbon nanotubes affixed to the resonator and coated with biopolymers configured to bind with the gaseous analyte. The gaseous analyte may be detected by operating the micromechanical piezoelectric resonator and functionalization layer in the presence of the gas, detecting a change in the resonant frequency of the resonator, and determining the concentration of the gaseous analyte from the change in resonant frequency. Finally, the layer of carbon nanotubes may be grown on the piezoelectric resonator by depositing a catalyst on a piezoelectric structure, heating the piezoelectric structure and the catalyst to enhance the growth of the carbon nanotubes, and growing the carbon nanotubes at growth sites on the piezoelectric structure.

### CROSS REFERENCE TO RELATED APPLICATIONS

This application claims the benefit of provisional U.S. Patent Application No. 61/146,866, filed Jan. 23, 2009.

### FIELD OF THE INVENTION

The present invention relates to chemical sensing, and is more specifically directed to the use of micromechanical devices to detect a gaseous analyte in a gas.

### BACKGROUND OF THE INVENTION

In recent years it has become desirable to use micromechanical devices for high performance chemical sensing. In particular, there is demand for miniature sensors for use in the detection of concentrations of potentially harmful and other chemicals. Micromechanical piezoelectric resonators are amenable for use in such sensing applications given their ability to be miniaturized and comparatively high operating frequencies.

Micromechanical sensing devices may have applications including national security, industrial emission monitoring, or medical diagnostics. In these applications there is an omnipresent need to increase the sensitivity, limits of detection, and response time of existing micromechanical sensors, while being conscious of size, portability, cost, and ease of monitoring.

### SUMMARY OF THE INVENTION

Aspects of the present invention are embodied in systems and methods for detecting a gaseous analyte in a gas. In one embodiment, a system for detecting a gaseous analyte in a gas includes a contour-mode piezoelectric resonator, and a functionalization layer affixed to the piezoelectric resonator. The functionalization layer is configured to bind with the gaseous analyte. The resonator has a first resonant frequency when the gaseous analyte is not bound to the functionalization layer and a second resonant frequency when the gaseous analyte is bound to the functionalization layer. The first and second resonant frequencies are different.

In another embodiment, a system for detecting a concentration of a gaseous analyte in a gas includes a micromechanical piezoelectric resonator, a layer of carbon nanotubes affixed to the resonator, and a plurality of biopolymers affixed to the layer of carbon nanotubes. The plurality of biopolymers is configured to bind with the gaseous analyte. The resonator has a first resonant frequency when the gaseous analyte is not bound to the plurality of biopolymers and a second resonant frequency when the gaseous analyte is bound to the plurality of biopolymers. The first and second resonant frequencies are different.

In yet another embodiment, a system for detecting a concentration of at least one gaseous analyte in a gas includes two or more micromechanical piezoelectric resonators, a layer of carbon nanotubes affixed to the each of the two or more resonators, and a plurality of biopolymers affixed to the layer of carbon nanotubes on each of the two or more resonators. The plurality of biopolymers on each of the two or more resonators is configured to bind with one of the at least one gaseous analyte. Each of the two or more resonators has a first resonant frequency when one of the at least one gaseous analyte is not bound to the plurality of biopolymers of the resonator and a second resonant frequency when one of the at least one gaseous analyte is bound to the plurality of biopolymers of the resonator. The first and second resonant frequencies are different.

In another embodiment, a method for detecting the concentration of a gaseous analyte in a gas involves operating a micromechanical piezoelectric resonator in the presence of the gas containing the gaseous analyte. The resonator is covered with a layer of carbon nanotubes affixed with a plurality of biopolymers configured to bind with the gaseous analyte. The resonator has a resonant frequency when the gaseous analyte is not bound to the plurality of biopolymers. The method further comprises detecting a change in the resonant frequency of the resonator and determining the concentration of the gaseous analyte in the gas from the change in resonant frequency.

In yet another embodiment, a binding property of single-stranded DNA may be determined by detecting a first resonant frequency of a resonator covered with a plurality of carbon nanotubes affixed with the single-stranded DNA, then exposing the resonator to a gas comprising a known gaseous analyte, then detecting a second resonant frequency of the micromechanical resonator, and then determining a difference between the first and second resonant frequencies of the micromechanical resonator.

In still another embodiment, a method for the large-scale integration of carbon nanotubes on a piezoelectric structure may include depositing a catalyst on the piezoelectric structure, heating the piezoelectric structure and the catalyst to provide a plurality of growth sites on the piezoelectric structure for carbon nanotubes; and growing a plurality of carbon nanotubes at the plurality of growth sites on the piezoelectric structure.

### BRIEF DESCRIPTION OF THE DRAWINGS

In order that the invention may be more fully understood, the invention will now be described by reference to the accompanying drawings, in which:

FIG. 1A depicts a side view of an exemplary system for detecting a concentration of a gaseous analyte in a gas in accordance with an aspect of the present invention;

FIG. 1B depicts an exploded view of an exemplary system for detecting a concentration of a gaseous analyte in a gas in accordance with an aspect of the present invention;

FIG. 2 depicts a flow chart of exemplary steps for detecting a concentration of a gaseous analyte in a gas in accordance with an aspect of the present invention;

FIG. 3 depicts an exemplary graph of the change in resonant frequency over time during the exposure of a resonator to a gas containing a gaseous analyte;

FIG. 4 depicts an exemplary graph of the change in mass of a resonator system dependent on the concentration of a gaseous analyte;

FIG. 5 depicts another exemplary graph of the change in resonant frequency over time during the exposure of a resonator to a gas containing a gaseous analyte; and

FIGS. 6A-6F depict exemplary steps for integrating carbon nanotubes onto a piezoelectric structure in accordance with an aspect of the present invention.

### DETAILED DESCRIPTION OF THE INVENTION

Aspects of the present invention are directed to systems and methods for detecting a gaseous analyte in a gas. The gaseous analyte may be any chemical element or compound present in a gas. Exemplary gaseous analytes include methanol, propionic acid, triemethyleamine, dinitrotoluene (DNT), and dimethylmethylphosphonate (DMMP).

An exemplary system is provided for detecting a gaseous analyte in a gas in accordance with one aspect of the present invention. As a general overview, the system includes a contour-mode piezoelectric resonator and a functionalization layer affixed to the resonator. Additional details of the disclosed system are provided below.

The contour-mode piezoelectric resonator may have a bottom and top electrode and a layer of piezoelectric material disposed between the bottom and top electrodes. The piezoelectric resonator may comprise any suitable piezoelectric material, including for example aluminum nitride (AlN), zinc oxide (ZnO), aluminum gallium arsenide (AlGaAs), gallium nitride (GaN), quartz, zinc-sulfide, cadmium-sulfide, lithium tantalate, lithium niobate, and other members of the lead lanthanum zirconate titanate family. In a preferred embodiment, the piezoelectric material comprises aluminum nitride. The top and bottom electrodes may comprise any conductive metal known to one of ordinary skill in the art, including for example platinum (Pt), aluminum (Al), molybdenum (Mo), tungsten (W), titanium (Ti), niobium (Nb), ruthenium (Ru), chromium (Cr), doped polycrystalline silicon, doped AlGaAs compounds, gold, niobium, copper, silver, tantalum, cobalt, nickel, palladium, silicon germanium, doped conductive zinc oxide (ZnO), and combinations thereof. In an exemplary embodiment, the resonator has a planar surface having two cantilevered ends. The resonator is sized such that an alternating current electric field applied across the top and bottom electrodes induces mechanical deformations in the plane of the resonator (i.e. contour-mode). A suitable contour-mode piezoelectric resonator for use with this or any other embodiment of the present invention is described by U.S. Patent Application Publication No. 2006/0290449 A1 to Piazza et al., which is incorporated herein by reference in its entirety.

A functionalization layer is affixed to the contour-mode piezoelectric resonator. The functionalization layer is configured to bind with the gaseous analyte being detected. In one embodiment, this binding may comprise adsorption by the functionalization layer of the gaseous analyte. In another embodiment, this binding is may comprise reacting with the gaseous analyte. The functionalization layer may be affixed to the top surface of the resonator to increase the surface area of the functionalization layer, thereby increasing the number of sites available to bind with the gaseous analyte.

The material comprising the functionalization layer may be selected from the group consisting of metal, metallic alloy, polymer, ceramic, carbon, nano-structure, or metallic and semiconducting nano-particles. Additionally, the functionalization layer may comprise electrically conductive materials, provided those materials are insulated from the top electrode of the resonator. Insulating layers for use with electrically conductive materials may include, for example, silicon dioxide or other dielectric materials. In one exemplary embodiment, the functionalization layer may comprise a layer of carbon nanotubes. The carbon nanotubes may, for example, consist of single-walled carbon nanotubes. The functionalization layer in this embodiment may further include biopolymers affixed to the layer of carbon nanotubes. The biopolymers may be selected based on their ability to bind with the gaseous analyte. The biopolymers may comprise, for example, single-stranded DNA configured to bind with the gaseous analyte.

In another exemplary embodiment, the functionalization layer may comprise fluoropolyol polymer (FPOL). A functionalization layer of FPOL may be used for the detection of gaseous analytes including nerve agents such as dimethylmethylphosphonate. In yet another exemplary embodiment, the functionalization layer may comprise a gold film affixed to the resonator for bio-sensing applications.

The contour-mode piezoelectric resonator and functionalization layer system has a resonant frequency before binding with any gaseous analyte. The resonant frequency is dependent on the dimensions of the resonator and the mass of the system, as will be understood by one of ordinary skill in the art. The binding of the functionalization layer with the gaseous analyte changes the mass of the system. Accordingly, the resonator and functionalization layer system will have a different resonant frequency after the functionalization layer binds with the gaseous analyte.

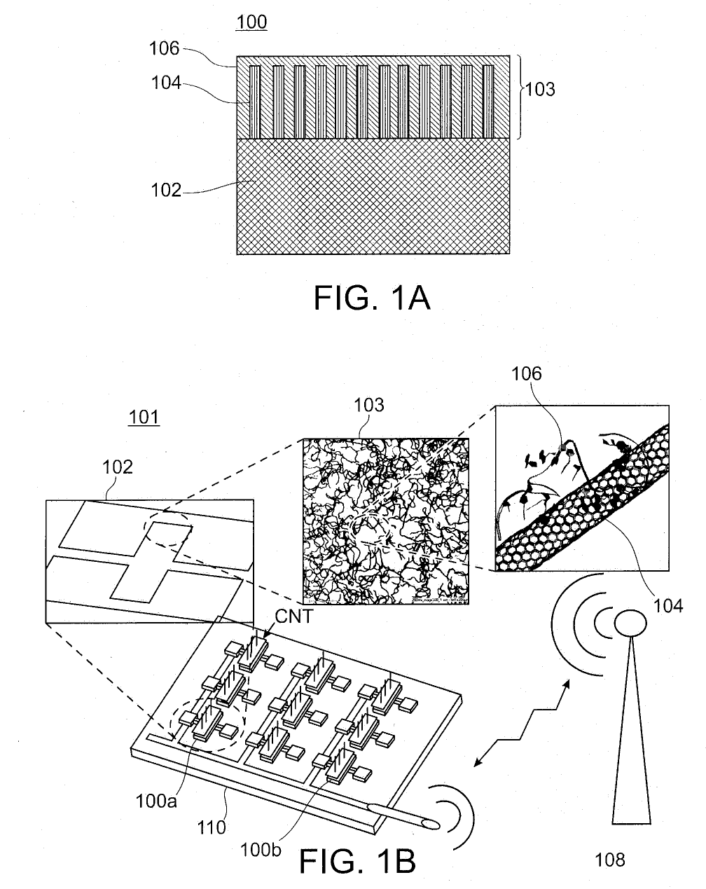

FIGS. 1A & 1B depict an exemplary system 100 for detecting a concentration of a gaseous analyte in a gas in accordance with aspects of the present invention. As a general overview, FIG. 1A depicts a system 100 including a resonator 102 coated with is a functionalization layer 103. The functionalization layer 103 may include a layer of carbon nanotubes 104 affixed with a plurality of biopolymers 106. FIG. 1B depicts a system 101 including a resonator array 110 configured to operate in conjunction with a wireless antenna 108. The array 110 has at least one resonator 102 coated with a functionalization layer 103. The functionalization layer 103 may include a layer of carbon nanotubes 104 affixed with a plurality of biopolymers 106. Additional details of the disclosed systems are provided below.

The resonator 102 may be a micromechanical piezoelectric resonator. The resonator 102 may comprise two electrodes and a layer of piezoelectric material disposed between them. The piezoelectric resonator may comprise any of the piezoelectric materials listed above by way of example. The electrodes may comprise any of the conductive materials listed above by way of example. In an exemplary embodiment, the resonator 102 is a contour-mode piezoelectric resonator, substantially as described above.

A functionalization layer 103 is affixed to the resonator 102. The functionalization layer may comprise a layer of carbon nanotubes 104. The carbon nanotubes may, for example, consist of single-walled carbon nanotubes. The layer of carbon nanotubes 104 may be affixed to the top surface of the resonator to increase the surface area available for binding with the gaseous analyte.

A plurality of biopolymers 106 is affixed to the carbon nanotubes. The biopolymers may be, for example, RNA, DNA, proteins, peptides, DNA, amino acids, mononucleotides, or polynucleotides. In a preferred embodiment, the biopolymers 106 are single-stranded DNA. The biopolymers 106 are selected based on their ability to bind with the gaseous analyte. The binding of the plurality of biopolymers with the gaseous analyte may comprise, for example, adsorption of the gaseous analyte by the plurality of biopolymers.

The system 100 has a resonant frequency before the plurality of biopolymers 106 bind with a gaseous analyte. The resonant frequency is dependent on the dimensions of the resonator 102 and the mass of the system 100. The binding of the plurality of biopolymers 106 with the gaseous analyte changes the mass of the system. Accordingly, the system 100 will have a different resonant frequency after the plurality of biopolymers binds with the gaseous analyte

An exemplary sensory system for detecting a concentration of a gaseous analyte in a gas is also provided in accordance with an aspect of the present invention. As shown in FIG. 1B, the sensory system 101 may generally include a resonator array 110 including two or more resonator systems 100a and 100b (for example) comprising a micromechanical piezoelectric resonator 102 coated with a functionalization layer 103. The functionalization layer may comprise a layer of carbon nanotubes 104 and a plurality of biopolymers 106, as described above with reference to resonator system 100.

The sensory system 101 may include an array 110 of resonator systems each comprising a single system 100. In one exemplary embodiment, different resonator systems 100 in the array 110 may include pluralities of different biopolymers 106, each biopolymer configured to bind with a different gaseous analyte.

Additionally, as noted above, the resonant frequency of each resonator system 100 will be dependent on the dimensions of the corresponding resonator 102 and the mass of each resonator system 100, as described above. In another exemplary embodiment of the present sensory system 101, different resonator systems 100 in the sensory system 101 may be configured to have different initial (i.e. pre-binding) resonant frequencies. The resonant frequency for each resonator system 100 will then change after the plurality of biopolymers 106 for each resonator system 100 has bound with the gaseous analyte, thereby changing the mass of the resonator system 100. The difference in the resonant frequency observed after binding for each resonator system 100 will further depend on the initial resonant frequency of the resonator system 100. For example, a resonator system 100 with a low resonant frequency may have a smaller change in resonant frequency after binding, whereas a resonator system 100 with a high resonant frequency may have a larger change in resonant frequency after binding. This difference may allow for a broader range of sensitivity to the concentration of a chosen gaseous analyte for sensory system 101.

The array 110 of resonator systems 100 may be configured to be operated wirelessly. In an exemplary embodiment, each resonator system 100 may be used as a passive radio-frequency (RF) transponder. A wireless antenna 108 may be configured to emit energy at a radio-frequency corresponding to the passive RF transponders in order to drive each resonator system 100 at its resonant frequency. A wireless antenna 108 may also be used to receive radio-frequency signals from each resonator system 100. Adaptation of the resonator 102 to receive and output radio-frequency signals will be understood by one of ordinary skill in the art from the description herein.

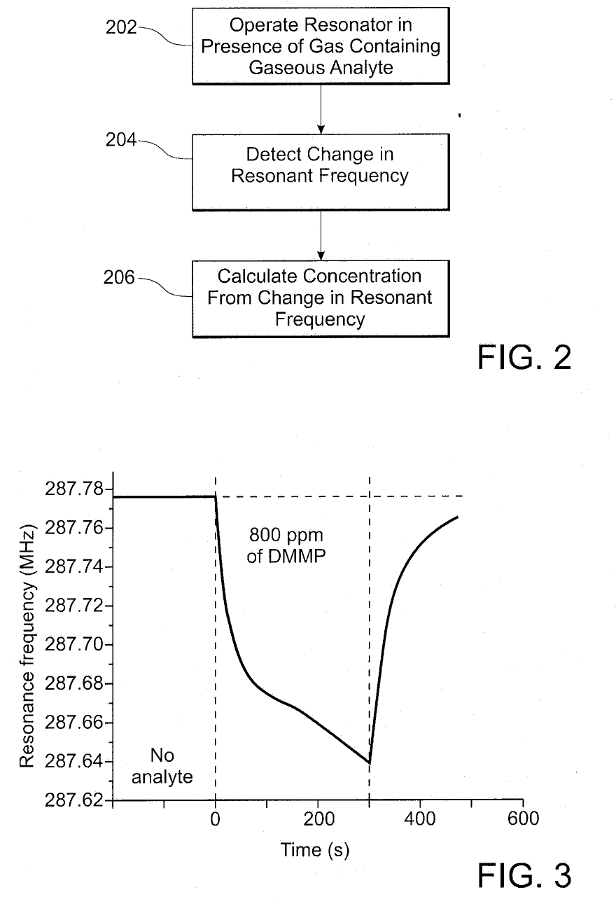

FIG. 2 is a flow chart depicting exemplary steps for detecting a concentration of a gaseous analyte in a gas in accordance with one aspect of the present invention. To facilitate description, the steps of FIG. 2 are described with reference to the system components of FIGS. 1A & 1B.

In step 202, a resonator 102 is operated in the presence of a gas containing the gaseous analyte. The resonator 102 may be any micromechanical piezoelectric resonator. In a preferred embodiment, the resonator 102 is a contour-mode piezoelectric resonator. The resonator 102 is covered with a layer of carbon nanotubes 104. Additionally, a plurality of biopolymers 106 are affixed to the layer of carbon nanotubes 104. The plurality of biopolymers may be selected based on their ability to bind with a particular gaseous analyte. The resonator system has a resonant frequency before binding with any of the gaseous analyte (a pre-binding resonant frequency).

The gas containing the gaseous analyte may be provided as a flow of gas adjacent to the resonator system 100. When operated, the piezoelectric material of the resonator 102 vibrates at the resonant frequency. In the presence of the gas containing the gaseous analyte, the plurality of biopolymers 106 may bind with molecules of the gaseous analyte, thereby increasing the mass of the resonator system 100. Because the resonant frequency of the resonator 102 is dependent on the mass of the resonator system 100, the binding of the gaseous analyte with the plurality of biopolymers 106 causes a change in the resonant frequency of the resonator 102.

In step 204, a change in the resonant frequency of the resonator 102 is detected. In an exemplary embodiment, the resonant frequency of the resonator 102 is monitored during the operation of the resonator 102 in the presence of the gas containing the gaseous analyte. FIG. 3 depicts an exemplary graph of the change in frequency of an exemplary resonator during exposure to a gas containing the gaseous analyte dimethylmethylphosphonate (DMMP). The change in resonant frequency of the resonator 102 may be charted as a function of the time of the exposure.

In step 206, the concentration of the gaseous analyte in the gas is calculated. The concentration of the gaseous analyte in the gas may be derived from the detected is change in resonant frequency of the resonator 102. In an exemplary embodiment, the extent of the change in resonant frequency depends on the mass sensitivity of the resonator and the amount of gaseous analyte bound to the resonator (the adsorbed mass). The sensitivity of the resonator system 100 to the adsorbed mass per unit area may be calculated using the following formula:

$$\frac{Δ f}{Δ ρ} = - \frac{{f}_{0}^{2}}{\sqrt{{E}_{0} \cdot {ρ}_{0}}} \cdot \frac{W}{T}$$

where Δf is the change in resonant frequency, Δρ is the mass per unit area adsorbed by the functionalization layer 103 (carbon nanotubes, functionalized carbon nanotubes, polymers, etc.) onto the resonator 102, f₀, is the pre-binding resonant frequency of the resonator, E₀ is the Young's modulus of the resonator system, ρ₀ is the mass density of the resonator system, W is the dimension setting the resonator resonance frequency, and T is the thickness of the piezoelectric film.

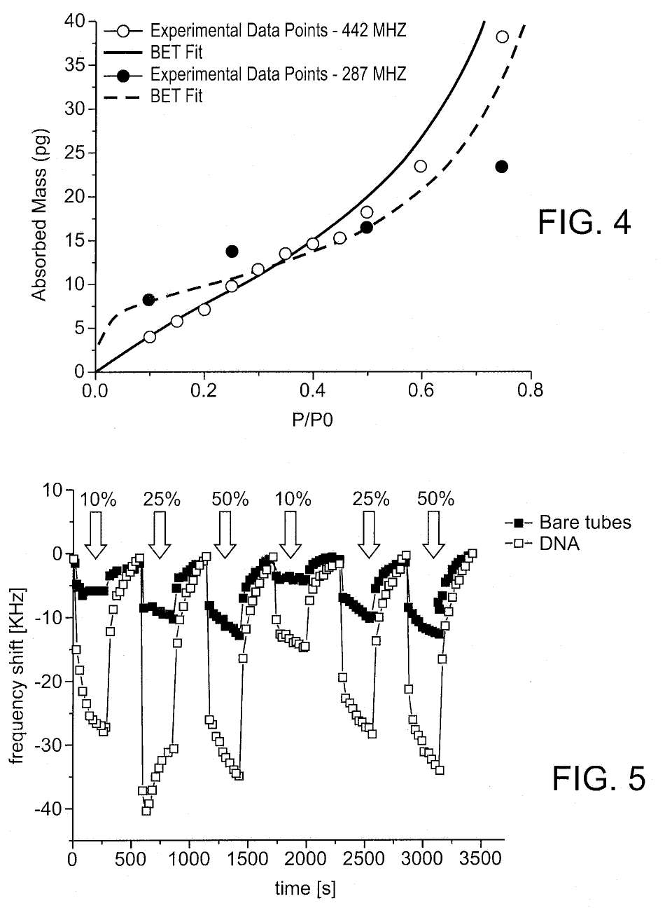

As noted above, the change in mass of the resonator system 100 is the result of particles of the gaseous analyte binding the with the plurality of biopolymers 106. As the number of adsorbed particles of the gaseous analyte is dependent on its concentration in the environment, the change in mass of the resonator system 100 will correspond to a concentration of the gaseous analyte in the gas. The relationship between the number of analyte molecules adsorbed by the sensitive layer and the analyte concentration in air may be determined experimentally for each resonator system by exposing the resonator system to gasses containing a known concentration of the gaseous analyte and then calculating the change in mass of the system from the change in resonant frequency. An exemplary graph of the change in mass of a resonator system (adsorbed mass) vs. the concentration of the gaseous analyte DMMP (P/P₀) is provided in FIG. 4.

After the resonant frequency of the resonator system 100 has changed due to binding with the gaseous analyte, the system 100 will optimally be reset to its pre-binding mass before detecting another concentration of a gaseous analyte. The system is reset by unbinding the particles of the gaseous analyte from the plurality of biopolymers 106. This unbinding may occur by exposing the system to a gas containing substantially high concentrations of non-reactive molecules, such as noble is gases. In an exemplary embodiment, the resonator system 100 may be reset by exposing the system 100 to argon gas.

The process of determining a binding property of single-stranded DNA with a chosen gaseous analyte will now be described in accordance with one aspect of the present invention. In an exemplary embodiment, the chosen type of single-stranded DNA to be analyzed is affixed to a layer of carbon nanotubes that has been affixed to a resonator, as described above with relation to system 100. A resonant frequency of the resonator may then be determined. The resonator is then exposed and operated in the presence of a gas having a known concentration of the chosen gaseous analyte (at a known volume). In a preferred embodiment, the gas may be comprised of a known concentration of the chosen gaseous analyte and a non-reactive gas, such as argon. Alternatively, the gas may consist entirely of the chosen gaseous analyte. Thereafter, a new resonant frequency of the system is determined. This process may be repeated with gases containing different known concentrations of the chosen gaseous analyte to determine the sensitivity of the selected strands of DNA to different concentrations of the known gaseous analyte.

A difference between the pre-exposure and post-exposure resonant frequencies may correspond to the ability of the single-stranded DNA to bind with the chosen gaseous analyte. A larger change in the resonant frequency of the resonator may correspond to a greater ability of the single-stranded DNA to bind with the chosen gaseous analyte. In an exemplary embodiment, the change in resonant frequency may be compared to the change in resonant frequency for a system containing only a layer of carbon nanotubes affixed to a resonator, without the accompanying plurality of single-stranded DNA, as set forth in FIG. 5. The difference between the change in resonant frequencies of the system having single-stranded DNA and the system lacking single-stranded DNA may provide additional information on the ability of the type of single-stranded DNA to bind with the chosen gaseous analyte. The change in resonant frequencies of the resonator may also be compared for exposures to different concentrations of the known gaseous analyte.

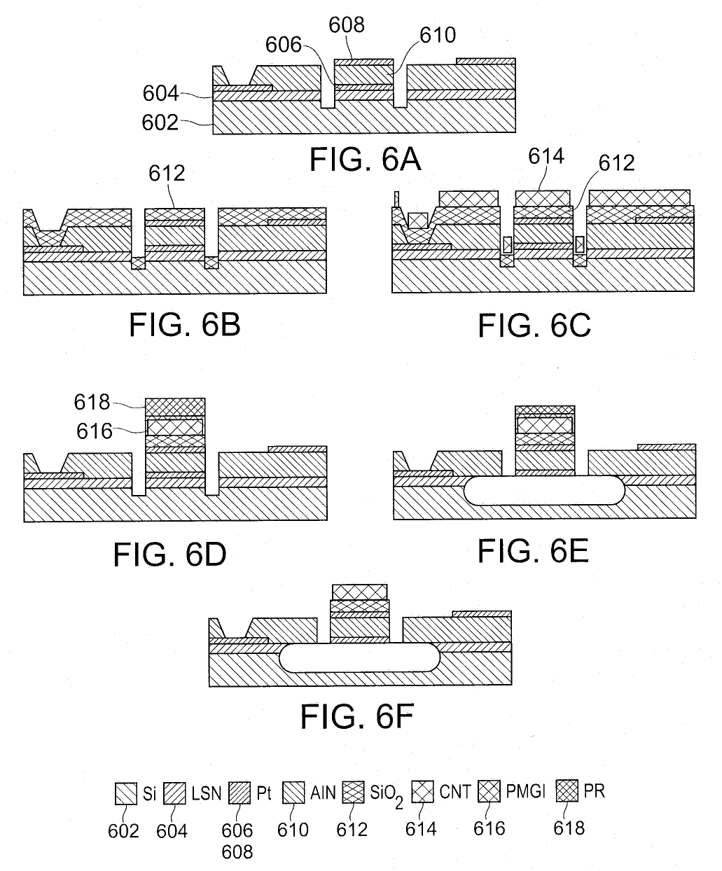

A process for growing carbon nanotubes on a piezoelectric structure will now be described with reference to FIGS. 6A-6F. FIGS. 6A-6F depict various stages in the formation of an the integration of carbon nanotubes onto a piezoelectric structure in accordance with one aspect of the present invention.

FIG. 6A depicts a piezoelectric structure formed on a substrate. In an exemplary embodiment, one or more piezoelectric structures may be formed on a silicon substrate 602 for large-scale fabrication of microelectromechanical or nanoelectromechanical devices. The microelectromechanical devices or nanoelectromechanical devices may, for example, comprise piezoelectric resonators. The piezoelectric structure may comprise a bottom electrode 606, piezoelectric material 610, and top electrode 608. The piezoelectric structure may be deposited on a surface 604 of the substrate 602, e.g., via sputtering. The substrate 602 may further include a coating of low-stress silicon nitride (LSN). The electrodes 606 and 608 may be comprised of a conductive metal including, for example, platinum. The piezoelectric material 610 may comprise aluminum nitride (AlN). The silicon substrate 602 and piezoelectric structure may then be lithographically patterned to form a piezoelectric structure having a desired shape or size. In one exemplary embodiment, the silicon substrate may be subdivided into smaller silicon chips, each chip containing a piezoelectric structure corresponding to one or more microelectromechanical or nanoelectromechanical piezoelectric resonators. The process of lithographically patterning the substrate in this step will understood by one of skill in the art from the description herein.

FIG. 6B depicts the piezoelectric structure with a catalyst provided thereon. In an exemplary embodiment, a catalyst such as silicon dioxide (SiO₂) may be sputtered onto the piezoelectric structure to serve as a seed layer 612 for the growth of carbon nanotubes. Additionally, a catalyst (not shown) may be deposited directly onto the piezoelectric material 610 or onto the layer of silicon dioxide 612. The catalyst may comprise, for example, an aqueous solution containing an iron salt. The piezoelectric material 610 is then steadily heated to the desired growth temperature. During this heating, the iron salt solution may be reduced by evaporation to elemental nanoscale iron grains. The nanoscale iron grains may serve as further growth sites for carbon nanotubes.

FIG. 6C depicts a layer of carbon nanotubes 614 on the piezoelectric structure and catalyst. In an exemplary embodiment, a layer of carbon nanotubes 614 is grown on the piezoelectric structure by chemical vapor deposition (CVD). During this process, carbon nanotubes are formed from the catalytic decomposition of hydrocarbon molecules. The hydrocarbon molecules may comprise, for example, methane or ethylene. This step is carried out at a sufficient high temperature to provide for the decomposition of the hydrocarbon molecules. The desired growth temperature may be about approximately 900° C. The growth of the layer of carbon nanotubes 614 may occur at the growth sites formed by the catalyst.

As discussed above, the silicon substrate 602 and piezoelectric structure may be subdivided into chips corresponding to piezoelectric resonators. These piezoelectric resonators, including the piezoelectric material 610 with the layer of grown carbon nanotubes 614, may be desirable for use as a chemical sensor as set forth above. In this application, it may be desirable to selectively pattern the layer of carbon nanotubes 614 on the piezoelectric resonators. Further steps for selectively patterning the layer of carbon nanotubes 614 on a piezoelectric resonator are provided herein.

FIG. 6D depicts the surface of the piezoelectric structure including the carbon nanotubes 614 covered with a protective layer 616. In an exemplary embodiment, the protective layer 616 may comprise polymethylglutarimide (PMGI) and a photoresistive layer 618. The layer of PMGI 616 may be advantageous in that it will not stick to the layer of carbon nanotubes 614 and may be removed without stripping the layer of carbon nanotubes 614 off of the piezoelectric structure.

The protective layer 616 may optimally be patterned such that only the layer of carbon nanotubes 614 on the desired areas of the surface of the piezoelectric resonators is protected. In an exemplary embodiment, the layer of carbon nanotubes 614 may be patterned to optimize the performance and/or the sensitivity of the piezoelectric resonators. To optimize the performance of the resonator, the layer of carbon nanotubes 614 may be patterned to remove carbon nanotubes that generate a high motional resistance to the vibration of the resonator. Layouts which optimize the performance of the resonator will be understood by one of skill in the art from the description herein. Alternatively, the layout of the carbon nanotubes 614 on the resonator may be chosen to maximize the sensitivity of the resonator of the system to a gaseous analyte. By way of example, if the piezoelectric resonator vibrates laterally, e.g., in the plane of the material, the resonator will be most sensitive to changes in the path of greatest displacement of the piezoelectric material 610 at resonance, which will occur at the peaks of the standing acoustic half wavelength. In this example, the layer of carbon nanotubes 614 may be selectively patterned to remain on the areas corresponding to the peaks of the standing half wavelength at resonance in order to maximize the sensitivity of the system. In another exemplary embodiment, the layer carbon nanotubes 614 may be selectively patterned to remain on the portion of the top surface of the piezoelectric resonator not including the top electrode, to increase the surface area of the carbon nanotube layer 614.

After the protective layer 616 has been selectively patterned, the resonator material may then be dry etched in a CF₄ based chemistry. The dry etching may remove unprotected carbon nanotubes and SiO₂ from the surface of the resonator material, while leaving unaltered the areas covered by the protective layer 616. The process of dry etching described in this step will be understood by one of skill in the art from the description herein.

FIG. 6E depicts a piezoelectric resonator released from the silicon substrate 602. In an exemplary embodiment, the resonator material is separated from the silicon substrate 602 or smaller silicon chips through dry isotropic etching with XeF₂. The process of dry isotropic etching with XeF₂ described in this step will be understood by one of ordinary skill in the art.

FIG. 6F depicts the protective layer 616 removed from the carbon nanotubes 614. The remaining layer of carbon nanotubes 614 may then be affixed with a plurality of biopolymers (not shown). In one exemplary embodiment, the solution containing the biopolymers may be dispensed onto the layer of carbon nanotubes on the surface of the piezoelectric resonator by a micropipette or an ink jet printer or any other suitable micro-arraying robot. The process may occur in a humid environment that provides sufficient time for the plurality of biopolymers to bind to the layer of carbon nanotubes. The resonator system may then be dried. In another exemplary embodiment, the plurality of biopolymers may be affixed to the layer of carbon nanotubes 614 by applying a solution comprising a plurality of biopolymers to the layer of carbon nanotubes. The solution may then be evaporated to leave the plurality of biopolymers affixed to the layer of carbon nanotubes 614.

As described above, the plurality of biopolymers may be, for example, RNA, DNA, proteins, peptides, DNA, amino acids, mononucleotides, or polynucleotides. In a preferred embodiment, the biopolymers are single-stranded DNA. The biopolymers to be affixed to the layer of carbon nanotubes 614 may be selected based on their ability is to bind with a gaseous analyte.

### EXAMPLE

In an example of the present invention, an array of contour-mode piezoelectric resonators were used to detect a gaseous analyte in a flow of gas. All resonators were affixed with a functionalization layer consisting of a layer of single-walled carbon nanotubes. Additionally, for some of the resonators, the single-walled carbon nanotubes were decorated with single-stranded DNA sequences. The array included separate contour-mode aluminum nitride piezoelectric resonators having resonant frequencies of 450 MHz and 287 MHz.

The functionalized resonators were then exposed to varying known concentrations of dimethylmethylphosphonate (DMMP). Specifically, each resonator was exposed to a flow of high purity argon gas that was combined with a flow of the desired analyte, DMMP. The resonator array was refreshed during the test by exposing the resonators to pure argon gas without the analyte. Each resonator underwent a cycle of differing known concentrations of DMMP followed by refresh phases.

Exposure of the resonators to the flow containing the analyte enabled the functionalization layer to adsorb molecules of DMMP. This resulted in a change in the resonant frequency of the resonators corresponding to the change in mass from the adsorption of the analyte. FIG. 3 depicts an exemplary graph showing a change in the resonant frequency of a resonator over time during the exposure of the resonator to the flow containing a concentration of DMMP of 800 parts per million. Before exposure, the resonator began with a resonant frequency of approximately 287.78 MHz. Exposure to the DMMP analyte increased the mass of the resonator, thereby decreasing the resonant frequency of the resonator to approximately 287.64 MHz.

FIG. 5 depicts a comparison of the frequency shift that was recorded for the resonators having a functionalization layer lacking the single-stranded DNA and the frequency shift recorded for the resonators having a functionalization layer including the single-stranded DNA. Those resonators having a functionalization layer including single-stranded DNA demonstrated an enhancement in the adsorption of the analyte, as demonstrated by their increase in change in resonant frequency for exposures to the same concentration of the analyte. After each frequency shift generated by exposure to the analyte, the resonators were exposed to a refresh phase returning the resonant frequencies to their pre-exposure levels.

After recording the change in resonant frequencies for the resonators, the adsorbed mass was calculated from the change in resonant frequency, using the methods described above. FIG. 4 depicts a plot of the adsorbed mass versus the concentration of DMMP at which that mass was adsorbed for both the resonators having 450 MHz resonant frequencies and those having 287 MHz frequencies. The data points were fitted with a curve corresponding to the mass sensitivity of the resonators. The fit line corresponding to the mass sensitivity can then be used to determine an unknown concentration of an analyte in a gas from a known adsorbed mass.

Although the invention is illustrated and described herein with reference to specific embodiments, the invention is not intended to be limited to the details shown. Rather, various modifications may be made in the details within the scope and range of equivalents of the claims and without departing from the invention. It will be understood by one of skill in the art from the description herein that one or more steps may be omitted and/or different components may be utilized without departing from the spirit and scope of the present invention.

What is claimed:

1. A system for detecting a gaseous analyte in a gas, the system comprising:
a contour-mode piezoelectric resonator; and
a functionalization layer affixed to the piezoelectric resonator, the functionalization layer configured to bind with the gaseous analyte, the system having a first resonant frequency when the gaseous analyte is not bound to the functionalization layer and a second resonant frequency when the gaseous analyte is bound to the functionalization layer, the first and second resonant frequencies being different.

2. The system of claim 1, wherein the functionalization layer comprises a layer of single-walled carbon nanotubes affixed to the piezoelectric resonator.

3. The system of claim 2, wherein the functionalization layer further comprises a plurality of biopolymers affixed to the layer of single-walled carbon nanotubes.

4. The system of claim 3, wherein the plurality of biopolymers is a plurality of single-stranded DNA.

5. The system of claim 1, wherein the functionalization layer is affixed to the top surface of the piezoelectric resonator.

6. A system for detecting a concentration of a gaseous analyte in a gas, said system comprising:
a micromechanical piezoelectric resonator;
a layer of carbon nanotubes affixed to the resonator; and
a plurality of biopolymers affixed to the layer of carbon nanotubes, the plurality of biopolymers configured to bind with the gaseous analyte, the system having a first resonant frequency when the gaseous analyte is not bound to the plurality of biopolymers and a second resonant frequency when the gaseous analyte is bound to the plurality of biopolymers, the first and second resonant frequencies being different.

7. The system of claim 6, wherein the resonator comprises a contour-mode piezoelectric resonator.

8. The system of claim 7, wherein the layer of carbon nanotubes is affixed to the top surface of the piezoelectric resonator.

9. The system of claim 6, wherein the layer of carbon nanotubes is a layer of single-walled carbon nanotubes.

10. The system of claim 6, wherein the plurality of biopolymers is a plurality of polynucleotides.

11. The system of claim 10, wherein the plurality of polynucleotides is a plurality of single-stranded DNA.

12. The system of claim 6, wherein the gaseous analyte is selected from a group consisting of methanol, propionic acid, triemethyleamine, dinitrotoluene, and demethyl methyl phosphonate.

13. A system for detecting a concentration of at least one gaseous analyte in a gas, said system comprising:
two or more micromechanical piezoelectric resonators;
a layer of carbon nanotubes affixed to the each of the two or more resonators; and
a plurality of biopolymers affixed to the layer of carbon nanotubes on each of the two or more resonators, the plurality of biopolymers on each of the two or more resonators configured to bind with a first of the at least one gaseous analyte, wherein each of the two or more resonators has a first resonant frequency when one of the at least one gaseous analyte is not bound to the plurality of biopolymers of the resonator and a second resonant frequency when the first gaseous analyte is bound to the plurality of biopolymers of the resonator, the first and second resonant frequencies being different.

14. The system of claim 13, wherein the first resonant frequency of one of the two or more resonators is different from the first resonant frequency of another one of the two or more resonators.

15. The system of claim 14, wherein the difference between the first and second resonant frequencies of each of the two or more resonators is dependent at least in part on the first resonant frequency of the resonator.

16. The system of claim 13, wherein the plurality of biopolymers on one of the two or more resonators is configured to bind with the first gaseous analyte, and the plurality of biopolymers on another one of the two or more resonators is configured to bind with a second of the at least one gaseous analyte.

17. The system of claim 13, wherein the layer of carbon nanotubes is a plurality of single-walled carbon nanotubes.

18. The system of claim 13, wherein the plurality of biopolymers is a plurality of polynucleotides.

19. The system of claim 18, wherein the plurality of polynucleotides is a plurality of single-stranded DNA.

20. A method of detecting a concentration of a gaseous analyte in a gas, the method comprising the steps of:
operating a micromechanical piezoelectric resonator in the presence of the gas containing the gaseous analyte, the resonator being covered with a layer of carbon nanotubes affixed with a plurality of biopolymers configured to bind with the gaseous analyte, the resonator having a resonant frequency when the gaseous analyte is not bound to the plurality of biopolymers;
detecting a change in the resonant frequency of the resonator; and
determining the concentration of the gaseous analyte in the gas from the change in resonant frequency.

21. The method of claim 20 wherein the resonator is a contour-mode piezoelectric resonator.

22. The method of claim 20, wherein the layer of carbon nanotubes is a plurality of single-walled carbon nanotubes.

23. The method of claim 20, wherein the plurality of biopolymers is a plurality of polynucleotides.

24. The method of claim 23, wherein the plurality of polynucleotides is a plurality of single-stranded DNA.

25. The method of claim 20 wherein the step of determining the concentration comprises:
determining the concentration of the gaseous analyte in the gas based at least in part on the magnitude of the change in frequency.

26. The method of claim 20, wherein the step of determining the concentration comprises:
determining a change in mass of the resonator based at least in part on the change in resonant frequency; and
determining the concentration of the gaseous analyte in the gas based at least in part on the change in mass of the resonator.

27. The method of claim 20, wherein the gaseous analyte is selected from a group consisting of methanol, propionic acid, triemethyleamine, dinitrotoluene, and demethylmethylphosphonate.

28. A method for determining a binding property of single-stranded DNA, the method comprising the steps of:
detecting a first resonant frequency of a resonator covered with a plurality of carbon nanotubes affixed with the single-stranded DNA;
then exposing the resonator to a gas comprising a known gaseous analyte;
then detecting a second resonant frequency of the micromechanical resonator; and
then determining a difference between the first and second resonant frequencies of the micromechanical resonator.

29. The method of claim 28, wherein the gas consists entirely of the known gaseous analyte.

30. The method of claim 28, wherein the gas comprises the known gaseous analyte and argon.

31. The method of claim 28, wherein the known gaseous analyte is selected from a group consisting of methanol, propionic acid, triemethyleamine, dinitrotoluene, and demethylmethylphosphonate.

32. A method for integrating carbon nanotubes onto a piezoelectric structure, the method comprising the steps of:
depositing a catalyst on the piezoelectric structure;
heating the piezoelectric structure and the catalyst to provide a plurality of growth sites on the piezoelectric structure for carbon nanotubes; and
growing a plurality of carbon nanotubes at the plurality of growth sites on the piezoelectric structure.

33. The method of claim 32, wherein the piezoelectric structure comprises piezoelectric material.

34. The method of claim 32, wherein the piezoelectric material comprises aluminum nitride.

35. The method of claim 32, further comprising the step of:
forming the piezoelectric structure on a substrate.

36. The method of claim 35, wherein the piezoelectric structure comprises a structure for one or more microelectromechanical or nanoelectromechanical devices on the substrate.

37. The method of claim 36, wherein the one or more microelectromechanical or nanoelectromechanical devices comprise one or more piezoelectric resonators on the substrate.

38. The method of claim 37, further comprising the step of:
dividing the substrate into chips corresponding to the one or more piezoelectric resonators.

39. The method of claim 32, wherein the catalyst is in the form of an aqueous solution.

40. The method of claim 39 wherein the aqueous solution comprises an aqueous iron salt solution.

41. The method of claim 32, wherein the plurality of growth sites on the piezoelectric structure comprise nanoscale iron grains.

42. The method of claim 32, wherein the catalyst comprises a layer of silicon dioxide.

43. The method of claim 32, wherein the step of heating the piezoelectric structure and the catalyst further comprises:
heating the piezoelectric structure and the catalyst up to approximately 900 degrees Celsius.

44. The method of claim 32, wherein the step of heating the piezoelectric structure and the catalyst further comprises:
heating the piezoelectric structure and the catalyst in an atmosphere containing hydrocarbons.

45. The method of claim 44, wherein the step of growing the plurality of carbon nanotubes is effected by catalytic decomposition of the hydrocarbons at the plurality of growth sites on the piezoelectric structure.

46. The method of claim 44, wherein the hydrocarbons comprise methane, ethylene, or a mixture of methane and ethylene.

47. The method of claim 32, further comprising the step of:
selectively removing the catalyst from regions of the piezoelectric structure.

48. The method of claim 32, further comprising the step of:
selectively removing the plurality of carbon nanotubes from regions of the piezoelectric structure.

49. The method of claim 38, further comprising the step of:
selectively removing the plurality of carbon nanotubes from regions of the one or more piezoelectric resonators.

50. The method of claim 49, wherein the plurality of carbon nanotubes is selectively removed from regions of the one or more piezoelectric resonators to optimize the sensitivity of the one or more piezoelectric resonators.

51. The method of claim 49, wherein the plurality of carbon nanotubes is selectively removed from regions of the one or more piezoelectric resonators to optimize the performance of the one or more piezoelectric resonators.

52. The method of claim 32 further comprising the step of:
affixing a plurality of biopolymers to the plurality of carbon nanotubes.

53. The method of claim 52, wherein the plurality of biopolymers is configured to bind with a gaseous analyte.

54. The method of claim 53, wherein the plurality of biopolymers is a plurality of polynucleotides.

55. The method of claim 54, wherein the plurality of polynucleotides is a plurality of single-stranded DNA.
